(19) United States
(12) Patent Application Publication
(10) Pub. No.: US 2026/0363608 A1
(43) Pub. Date: Nov. 19, 2026

(54) METHOD AND SYSTEM FOR ASSESSING INTRA-ABDOMINAL HEALTH

(71) Applicant: GI Matters Pty Ltd, Sydney (AU)

(72) Inventors: Pramesh KOVOOR, St Ives (AU); Michael Anthony BARRY, Werrington (AU); Joshua George KOVOOR, St Ives (AU)

(21) Appl. No.: 19/126,520

(22) PCT Filed: Nov. 2, 2023
(86) PCT No.: PCT/AU2023/051109

§ 371 Date: May 1, 2025

(30) Foreign Application Priority Data
Nov. 2, 2022 (AU) 2022903273

(51) Int. Cl. A61B 5/00; A61B 5/053

(52) U.S. Cl. CPC A61B 5/4238; A61B 5/053; A61B 5/4255; A61B 5/6823; A61B 5/7271

(57) ABSTRACT

A method of detecting function or alteration in function of the stomach, small intestine, or peritoneum, and especially gastrointestinal motility, of a patient, comprising steps of: applying electrodes to opposite sides of an abdomen of the patient, the electrodes including at least one electrode for supplying electric current to the abdomen and at least one electrode for receiving electric current along a current path through the abdomen; providing an alternating current signal to the electrodes; and determining or measuring conductivity of the abdomen along the current path between the electrodes, wherein changes in the measured or determined conductivity of the abdomen over a period of time provide an indication of function or dysfunction of the stomach, small intestine, or peritoneum. The disclosure also provides an associated system of assessing function or alteration in function of the stomach, small intestine, or peritoneum, and especially gastrointestinal motility, of a patient.

### TECHNICAL FIELD

The present disclosure relates to a method and a system of assessing intra-abdominal health, and particularly of evaluating function of the stomach, small intestine, and peritoneum. The disclosure especially relates to a method and system of assessing gastrointestinal motility and function of the gastrointestinal tract within this region of a patient. It will therefore be convenient to describe the present disclosure in the context of a method and system for assessing gastrointestinal motility and peritoneal function.

The method and system of the present disclosure is particularly suited for use with patients who may be suffering from gastrointestinal or peritoneal dysfunction. This state can be quite common, for example, in patients on medications or treatment that affects gastrointestinal function, such as GLP-1 receptor agonists (e.g., semaglutide) or analgesic agents (e.g., opioids), or patients with serum electrolyte imbalance, patients recovering from an operative or non-operative intervention, in patients suffering from disease states (e.g., diabetes mellitus, ascites) and/or patients undergoing continuous ambulatory peritoneal dialysis. It will be appreciated, however, that the method and system of this disclosure is not limited to these applications and may also be employed for evaluating function within this region more generally.

### BACKGROUND ART

Any reference to background art herein, including to any documents, is intended to facilitate an understanding of the present disclosure only and shall not be considered as an admission that such background art is widely known or forms part of the common general knowledge in the relevant field in Australia or in any other country.

The human abdomen is the section of the human body between the thorax and pelvis. The abdomen contains a range of structures, but very importantly contains the organs and gastrointestinal tract that function together as the gastrointestinal system. Dysfunction within the human abdomen, and especially of the gastrointestinal system, can be transiently or enduringly present in various pathological states (e.g., diabetes mellitus), as a side-effect of medications, and/or as a by-product of operative and non-operative intervention. A notable form of gastrointestinal dysfunction is ileus, which is a diminished or uncoordinated movement of the gastrointestinal tract. The gastrointestinal tract essentially comprises a long tube with an entry at the stomach and an exit at the anus. Food moves through the gastrointestinal tract under the action of peristalsis, which is a series of wave-like muscle contractions in the walls of the gastro-intestinal tract. If there is lack of movement through any part of the tract, then all parts upstream of, or ‘above’, that part will also cease to engage in peristalsis. The parts located downstream of, or ‘below’, the place where there is a lack of movement may continue to display peristalsis. The lack of movement can be the result of medications that modify gastrointestinal function, disease states such as diabetes, serum electrolyte imbalance, a blockage (tumour, adhesion, intussusception), an inadequate blood supply (thrombo-embolic infarction), an inflammation (diverticulitis), an infection (appendicitis), or the result of an operation, after which the gut may be “stunned” for a time.

The current techniques of evaluating function and dysfunction of the stomach, small intestine, and peritoneum of a patient can be divided into patient history, physical examination, vital sign observations, laboratory tests, and investigative methods ranging in invasiveness from imaging modalities to exploratory surgery. History taking will typically identify symptoms that the patient may be experiencing, such as abdominal pain, nausea, vomiting, or change in bowel habits.

Physical examination usually elicits clinical signs, such as abdominal distension, shifting dullness, and the auscultation of bowel sounds. Bowel sounds are associated with peristalsis but may vary significantly in their frequency and pitch even in a healthy patient. Bowel sounds are intermittent in post-operative patients at the best of times. The accepted method is for the clinician to listen for ten minutes at a time in each hour to the four quadrants of the abdomen, using a stethoscope. This is usually impractical, however, due to other duties required of the clinician and the associated variability in bowel sound presence and location. Auscultation is often performed with suboptimal listening times, which can lead to extended fasting times for patients or (occasionally) to ingestion of food before peristalsis is confirmed.

Investigative methods vary in their utility depending on clinical circumstances. Imaging modalities are useful when there is suspected pathology from the patient history and/or physical examination, particularly in emergency situations. However, no bedside imaging modality has yet been found to be suitable for regular, everyday use in evaluating gastrointestinal dysfunction or variation in function. One such modality that has been tested in this regard is portable ultrasound. However, this is often found to be of limited value, particularly given the confounding effect that bowel gas and obesity can have on the image acquisition and quality.

Autonomous methods for detecting bowel sounds have not proven successful as artefactual sounds picked up by a stethoscope or microphone are frequent and loud in comparison to true gut sounds, and they often sound similar. Patients also frequently move and turn, causing further artefacts which may obscure or ‘drown out’ any true gut sounds. A further issue or difficulty with recording sounds in a hospital ward are privacy and confidentiality concerns, with the result that, to date, there is no automated method for bowel sound detection in common use. The ‘gold-standard’ methods for determining gut motility involve the use of radioactive tracers and gamma cameras with scintigraphy. These are used primarily in research, but are rarely suitable for use in clinical practice post-operatively given their requirements for resources, the use of radioactive materials, and the necessity for the patient to swallow the tracer at a time when function of the gut has not been established.

A few researchers have proposed methods of assessing gut motility that require the patients to ingest a sizeable quantity (e.g., several hundred mL) of high-salt content “contrast” medium to provide an indication of stomach emptying. This is not appropriate for post-operative patients due to the necessity of ingesting large amounts of the salty liquid, particularly when return of gastrointestinal function may not have been confirmed yet in the early postoperative phase. The contrast media is additionally rather difficult for patients to swallow due to the salty taste, and this does not add to the attractiveness of the method. This is particularly so given that this increases the risk of nausea and vomiting in a patient population where this risk is already high (i.e., patients who have undergone surgery). Aspiration pneumonia is a notable cause of morbidity and mortality after general surgery and the risk of this increases with vomiting.

Other researchers have in the more recent past described auscultation methods that rely on piezoelectric transducers (i.e., as microphones or acoustic energy sensors) to perform signal processing for detection of gastrointestinal motility and irritable bowel syndrome. While this work has been in progress for some time, as exemplified in United States Patent Application Publication No. US 2008/0253535 A1, it has not yet matured to be commercially available.

Following the rapid development of electrocardiography (ECG), some attempts have been made to employ cutaneous electrogastrography (EGG) as a non-invasive technique to detect and record gastric myoelectrical activity via electrodes placed on the abdominal surface. That is, the electrodes are adapted to detect and acquire the natural electrophysiological data from a subject under investigation. An example is described in United States Patent Application Publication No. US 2018/0317800 A1. Importantly, it is not yet settled which myoelectrical variable can be reliably recorded by EGG or how EGG correlates with gastric motility and gastric emptying.

In other work, a system for monitoring colon motility by electrode pairs placed in the pelvic region of a subject on each of posterior lateral sides (buttocks) of the body is described in the United States Patent Application Publication No. US 2020/0253535 A1. Importantly, that system is not suited to detect or assess the gastrointestinal motility of a patient in the stomach and/or pyloric region.

It would be desirable to provide a new method and system of assessing intra-abdominal or gastrointestinal health, and especially gastrointestinal motility, of a patient. More particularly, it would be desirable to provide a method and system of evaluating function and dysfunction of the stomach, small intestine, or peritoneum, and especially gastrointestinal motility or alteration of function in this region of a patient. In this context, it would be desirable to provide a method and system that may assess peristalsis at the stomach and/or pyloric region and does not rely on analysis of sound.

### SUMMARY

According to one aspect, the present disclosure provides a method of assessing intra-abdominal health, particularly of evaluating function or dysfunction of the stomach, small intestine, or peritoneum, and especially of assessing gastrointestinal motility or an alteration of function within this region, comprising:
applying at least two electrodes to an abdomen of the patient, and preferably to opposite sides (e.g., the front/anterior and rear/posterior sides) of the abdomen;
providing a current signal to the electrodes for supplying electric current to the abdomen; and
determining or measuring conductivity of the abdomen along the current path between the electrodes;
evaluating changes in the conductivity of the abdomen determined or measured over a period of time, wherein the changes in the conductivity of the abdomen over a period of time provide an indication of intra-abdominal or gastrointestinal function, and especially function of the stomach, small intestine, or peritoneum.

The at least two electrodes include at least one electrode for supplying electric current to the abdomen and at least one electrode for receiving electric current along a current path through the abdomen. For the purposes of description, the at least one electrode for supplying electric current to the abdomen will be referred to as a “driving electrode” and the at least one electrode for receiving electric current along a current path through the abdomen will be referred to as a “sensing electrode”. It will be appreciated that the driving electrode and the sensing electrode may be essentially identical. Further, when connected in a circuit with an alternating current (AC) source, the reference to a “driving” electrode or a “sensing” electrode in the method or system of this disclosure is purely notional in the sense that whichever electrode of the pair of electrodes is operating as the “driving” electrode or as the “sensing” electrode may simply vary (i.e., alternate) with time.

In an embodiment, the step of applying the electrodes to the abdomen of the patient, preferably to opposite sides of the abdomen, is adapted to maximise the current flow through the gastrointestinal tract. The step may include applying the electrodes in a region of the patient's stomach, the pyloric antrum, duodenum and/or at a level of the patient's kidneys. In the context of the present disclosure, it will be appreciated by persons skilled in the art that the exit from the stomach (i.e., the pylorus) has a well-defined anatomical position (i.e., the transpyloric plane). This is distinct from other parts of the digestive tract, which are much more mobile. Further, as it is at the top of the digestive tract, any blockage in any part of the digestive tract will cause the pylorus to cease passing material into the duodenum. The present disclosure can thus use this position to monitor gut motility. If there is peristalsis occurring at the level of the pylorus, then one may deduce that there must be no downstream issue with gut motility.

Thus, by way of example, the electrodes may be arranged to measure the conductivity across the stomach and/or the duodenum at the pyloric sphincter. It will be noted that the conductivity measurements will typically vary from individual to individual depending on their personal physiology, but also upon their intake of food and liquid. Thus, the system and method of the disclosure involve monitoring an individual over a period of time to track changes in the conductivity measurements for that individual, rather than for the particular absolute values to be measured or determined. Changes in conductivity measured or determined by the electrodes may be due to fluid movement in the body. Where changes are detected across the pyloric sphincter, one may deduce that the changes occur as a result of fluid shifting across the pyloric sphincter. This may thus indicate peristalsis which can be flagged for confirmation by medical personnel.

In an embodiment, the step of providing the current signal to the electrodes, preferably an alternating current signal, includes providing the signal intermittently or at intervals over a period of time, and the step of determining or measuring conductivity of the abdomen along the current path between the electrodes includes determining or measuring the conductivity on a periodic basis over the period of time. In this regard, the step of determining or measuring the conductivity is performed via a meter or sensor when the signal is applied to the electrodes.

In an embodiment of the disclosure, the step of applying the electrodes to the abdomen of the patient, and preferably to opposite sides of the abdomen, comprises applying one electrode on a first side (e.g., a rear or posterior side) of the abdomen and applying at least two other electrodes spaced apart from one another by a distance that is preferably in a range of about 100 mm to about 500 mm on a second side (e.g., a front or anterior side) of the abdomen. In this case, each of the front electrodes will typically receive electric current from the rear electrode along a partially divergent or separate current path through the abdomen. Preferably, the step of determining or measuring the conductivity is performed between the rear electrode and each of the front electrodes (e.g., via meter or sensor) sequentially. That is, the electrode pairs (i.e., the rear electrode with each front electrode) are interrogated sequentially to determine or measure the conductivity along the separate current paths.

In an embodiment of the disclosure, the step of applying the electrodes to the abdomen of the patient, and preferably to opposite sides of the abdomen, comprises applying at least two (driving) electrodes spaced apart from one another by a distance that is preferably in a range of about 100 mm to about 500 mm on a first side (e.g., a rear or posterior side) of the abdomen, and applying at least two (sensing) electrodes spaced apart from one another by a distance that is preferably in a range of about 100 mm to about 500 mm on a second side (e.g., a front or anterior side) of the abdomen. In this case, each electrode pair (e.g., each of the rear electrodes paired with a respective one of the front electrodes) is adapted to measure the conductivity along a separate, or at least partially separate, current path through the abdomen.

In past work, one researcher (Sutton) used a Kelvin (four-terminal) impedance measurement to acquire stomach impedance across one discrete current path. The present disclosure involves measuring or determining conductivity (the reciprocal value) and uses a number of multi-terminal measurements (e.g., two, three, or four) instead of one four terminal measurement. The anatomical locations of interest in this disclosure are also different to those investigated by Sutton. In addition, the method and system of this disclosure are not reliant on the use of a contrast medium (e.g., a low conductivity contrast medium) that a patient was required to ingest for the Sutton work, which—as noted above—is often medically inadvisable for post-surgical patients. With the system and method of the present disclosure, the patient does not need to consume contrast media for the investigation. Instead, the present method and system rely on temporal differentials in signals between different conductivity paths to assess the gastrointestinal motility and/or intra-abdominal function. In particular, the present method and system employ different patterns, namely: 1. opposing differentials in signals between different conductivity paths at the same time, and 2. changes in conductivity along individual conductivity paths over time, to assess function or alterations in function (or conversely to refute dysfunction) of the stomach, small intestine, or peritoneum.

In an embodiment of the disclosure, the step of providing the current signal involves or includes providing the current signal intermittently or at intervals to each of the electrodes in a sequence, being temporally offset from one another by a short time, e.g., of less than one second, and preferably in a range of 10 ms to 500 ms. In this way, the current from one (driving) electrode is separately and sequentially received at each of two other (sensing) electrodes in the pairs along two respective current paths and the respective conductivities of those current paths are determined or measured. Current from another of the electrodes is then likewise transmitted and received in a sequence or temporally offset at each of the two other (sensing) electrodes along two further current paths and the respective conductivities of those current paths are then similarly determined or measured.

Thus, in an embodiment of the disclosure, the step of determining or measuring the conductivity of the abdomen includes determining or measuring the conductivity between each pair of electrodes (e.g., comprising a ‘driving’ electrode and a ‘sensing’ electrode) on a periodic basis over the period of time. The period of “dwell” on each current path (i.e., the time or period over which current is transmitted) between the respective pairs of electrodes is preferably relatively short; e.g., in a range of about 1 to 10 cycles of a frequency of the alternating current. For alternating current supplied at 50 Hz, the “dwell” is preferably in a range of about 20 ms to 200 ms. Preferably, each of the respective electrode pairs is energised, or has the current signal applied to them, in a predetermined sequence. The whole sequence or “loop” through the electrode pairs is preferably repeated without pause.

In an embodiment of the disclosure, the step of providing the signal comprises providing an alternating current signal. It will be appreciated, however, that the method and system of this disclosure are not limited to use of an alternating current signal and that other signal forms may be used. For example, a direct current signal and/or a range of tailored signal forms are feasible.

In an embodiment, the step of providing the current signal includes providing both a low frequency alternating current signal and a high frequency alternating current signal to or between the electrodes for determining or measuring both a low frequency conductivity of the abdomen along the current path between the electrodes and a high frequency conductivity of the abdomen along the current path between the electrodes.

In an embodiment of the disclosure, the step of determining or measuring the conductivity of the abdomen includes determining or measuring conductivity along the current path(s) for both a low frequency alternating current signal and a high frequency alternating current signal, with an overall conductivity of the abdomen along the current path(s) being based on conductivity measurements for both the low frequency and the high frequency signals. Thus, determining or measuring conductivity along the current path(s) is via a dual-frequency arrangement having a low frequency alternating current signal and a high frequency alternating current signal. By employing both low frequency current signals and high frequency current signals, dual frequency conductivity disparity can be used to assess movement of the gastrointestinal tract, the presence of swelling of the wall of the gastrointestinal tract, or alteration of the characteristics of ascitic fluid or function of the peritoneum. The high frequency conductivity measurement provides information on the tissue between the two given electrodes, whereas the low frequency conductivity measurement provides information on the fluid outside of cells that is being moved around by peristalsis across the same region between the two electrodes.

In an embodiment of the disclosure, the step of providing an alternating current signal to the at least one electrode pair includes providing a low frequency alternating current signal of less than about 5 kHz, preferably in the range of about 200 Hz to about 2 kHz, to the at least one electrode pair. Thus, the step of providing a dual-frequency, alternating current signal to each electrode pair includes a low frequency AC signal of less than about 5 kHz, preferably from about 200 Hz to about 2 KHz.

In an embodiment of the disclosure, the step of providing the alternating current signal to the at least one electrode pair includes providing high frequency alternating current of more than about 5 kHz, preferably in a range of about 5 kHz to about 50 kHz, and more preferably of about 20 kHz, to the at least one electrode pair. Thus, the step of providing a dual-frequency, alternating current signal to each electrode pair includes a high frequency AC signal of more than about 5 kHz, preferably from about 5 kHz to about 50 kHz This high frequency range provides the ability to assess extracellular fluid levels.

In an embodiment of the disclosure, the step of determining or measuring the conductivity of the abdomen across all of the electrodes is performed at least 5 times per second, preferably multiple times per second, and more preferably in the range of 5 times to 30 times per second.

In an embodiment of the disclosure, the step of providing an alternating current signal includes providing the current signal to the at least one driving electrode at a current of less than or equal to about 10 mA. Currents of up to 10mA can be acceptable at relatively high signal frequencies, such as above 10 kHz. On the other hand, currents may be considerably lower, e.g., preferably less than or equal to about 100 μA, as long as the signal frequency is also low, e.g., up to about 800 Hz. It is noted that, at higher frequencies (e.g., over 1 kHz) currents may be larger without stimulating neuro-muscular junctions in the area of the electrode, which can lead to irritation or discomfort for the patient. Smaller currents are therefore often preferred, but there is a trade-off between precise measurements of conductivity (usually requiring higher current) and avoidance of stimulation (usually requiring lower current). The IEC 60601-1 standard for medical electrical equipment provides guidance on suitable currents and frequencies that are safe for use with patients.

In past work, Sutton used a single exciting frequency (100 kHz) for impedance measurements. By contrast, the method and system of the present disclosure desirably use a dual-frequency arrangement with both a low frequency signal and high frequency signal for measuring conductivity. This provides an ability to measure extracellular fluid levels which previous measurements could not do, and at the same time provides a baseline measurement for comparing conductivities along each of the current paths. Previous work by Sutton involved a continuous exciting current of 4 mA at 100 kHz. By contrast, the present disclosure uses regulatory permitted pulsed currents at both low frequency and high frequency designed to avoid neuromuscular stimulation.

In an embodiment, the method of the present disclosure includes characteristics and employs techniques for addressing three main artefactual causes of conductivity change in the abdomen, namely (i) respiration (or speaking or coughing), (ii) gross body movement (sitting up, turning over, etc.), and (iii) heartbeat (i.e., the aorta pulsates and causes a measurable conductivity change). Starting with heartbeat, this can be trapped or captured by noting heart rate from an ECG signal. This is typically around the 1 Hz to 3 Hz level and can be functionally eliminated by determining or measuring conductivity of the abdomen across the electrodes, and averaging at 0.3 Hz. Gross body movement can be accounted for using a three-axis accelerometer placed on the patient (e.g., at an electrode position). If the accelerometer indicates motion above a threshold level (e.g., 0.1g above or below an ambient level representing a resting body), the conductivity measurements may be ignored until the accelerometer readings return to the ambient or ‘normal’ level of a resting body. The respiration artefacts are more difficult to account for or ‘quarantine’ or ‘trap’. Because respiration is a large signal with a period of about 2 to 5 seconds, a boxcar detector can be used to synchronise with the respiration and invert the change. This removes most of the effects, though respiration rate variation and/or speaking may still create anomalies. During speech the patient's diaphragm will move in irregular ways, with occasional rapid changes in conductivity (e.g., at the end of sentences or in pauses) interspersed by slow, measured diaphragm movements which produce almost no conductivity change. It is anticipated that such artefacts may be resolved in a similar manner as for gross body movement—i.e., by a speech detector that can indicate when to ignore the conductivity measurements.

According to another aspect, the disclosure provides a system for assessing or evaluating function, alteration in function, or dysfunction of the small intestine, stomach, and peritoneum, and especially for assessing or evaluating gastrointestinal motility, of a patient. The system comprises: at least one pair of electrodes adapted for application to an abdomen of the patient, preferably to opposite sides of the abdomen, for supplying and receiving an electric current along a current path through the abdomen; a current source for providing a current signal to at least one electrode of the electrode pair; a meter or a sensor for determining or measuring a conductivity of the abdomen along the current path between the electrodes; and a processor for analysing and/or evaluating the conductivity of the abdomen determined or measured by the meter or sensor over a period of time to provide an assessment of gastrointestinal health, and particularly to detect alterations in function of the stomach, small intestine, or peritoneum. To this end, changes in the conductivity of the abdomen in a region of the stomach, small intestine, or peritoneum over a period of time can provide an indication of gastrointestinal motility and/or function within this region.

According to still another aspect, the disclosure provides a device for use in detecting function/dysfunction of a stomach, small intestine, or peritoneum of a patient, and especially gastrointestinal motility, the device comprising:
a current source adapted for providing or applying an electric current signal to or between at least one pair of electrodes adapted for application to an abdomen of the patient;
a meter or a sensor for determining or measuring a conductivity of the abdomen along the current path between the electrodes; and
a processor for analysing the conductivity of the abdomen that is determined or measured by the meter or sensor over a period of time to provide an assessment of gastrointestinal motility or dysfunction.

In an embodiment of the disclosure, the current source is adapted for providing or applying an alternating current signal to or between the pair of electrodes. It will be appreciated, however, that this disclosure is not limited to use of an alternating current signal and that other signal forms may be used. For example, a direct current signal and/or a range of tailored signal forms are feasible. At least one of the electrodes will typically supply the electric current to the abdomen and at least one of the electrodes will receive the electric current along the current path through the abdomen.

In an embodiment of the disclosure, the system or device includes a memory, especially a digital memory or storage device, for recording or storing the determined or measured conductivities of the abdomen along the current path(s) between the at least one pair of electrodes over a period of time. In this way, the memory may store a history of use of the system for one or more patients. The memory may, for example, comprise a random access memory of the processor or, alternatively, a flash drive or some other storage device. The system or the device is preferably connectable to a display or to a printer, e.g., physically, directly, or wirelessly, in order to display or print the results of an assessment or evaluation of the function, alteration in function, or dysfunction of the stomach, small intestine, or peritoneum for one or more patients, and especially of the gastrointestinal motility recorded over a period of time.

In an embodiment of the disclosure, the system and the device may be adapted to communicate with and/or to interact with one or more remote or mobile devices. In this regard, the system and/or device of the disclosure preferably includes a software application for supporting access to the analysis and the measurements of the system or device by one or more individual remotely. For example, the software application may accessible or operable via a mobile telecommunications device (“mobile device”), such as a smartphone or tablet. In this way, the analysis and/or measurements of the system from one or more patients could be made available to or communicated to a medical professional located remote from the patient(s).

In an embodiment of the disclosure, the current source is adapted to provide a low frequency alternating current signal, preferably of less than about 5 kHz, and more preferably in a range of about 200 Hz to 2 kHz, to the at least one driving electrode. The low frequency alternating current signal is preferably provided within a dual-frequency arrangement.

In an embodiment, the current source is adapted to provide a high frequency alternating current signal, preferably of more than about 5 kHz, more preferably in the range of about 5 kHz to about 50 kHz, and even more preferably of about 20 kHz, to the at least one driving electrode. The high frequency alternating current signal is preferably provided within a dual-frequency arrangement.

In an embodiment of the present disclosure, the meter or sensor is adapted to determine or measure the conductivity of the abdomen along the current path between the electrodes both for the low frequency alternating current signal and for the high frequency alternating current signal, e.g., in a dual-frequency operation. In this way, a “normal” or overall conductivity of the abdomen along the current path may be based on the conductivity measurements for both the low frequency and high frequency signals. Preferably, the current source is adapted to provide the alternating current signal to the at least one pair of electrodes at a current of less than or equal to about 10 mA for a high frequency AC signal, and less than or equal to about 100 μA for a low frequency AC signal, with reference to the IEC 60601-1 standard, as discussed above.

In an embodiment, the sensor is adapted to determine or measure conductivity of the abdomen between the at least one pair of electrodes multiple times per second, and desirably at least 5 times per second. To this end, the sensor desirably interrogates or samples the electrodes at a rate in the range of 5 times to 30 times per second. By sampling or interrogating the electrodes to measure the conductivity multiple times per second, it is possible to eliminate the influence of other biological movements or signals, such as cardiac signals, from the measurements. In this regard, samples taken at a rate of five times per second enable the cardiac signal to be addressed, although sampling at higher rates deal with it better; e.g., 10 times per second or 20 times per second. The other biological signals from humans are typically of lower frequency; e.g., respiration is approx. 0.2 Hz.

In an embodiment, the at least one pair of electrodes comprises at least four electrodes, including at least two electrodes adapted to be applied spaced apart from one another on a first (e.g., a rear or posterior) side of the abdomen and at least two electrodes adapted to be applied spaced apart from one another on a second (e.g., a front or anterior) opposite side of the abdomen. Each of the sensing electrodes receives electric current from each of the driving electrodes along a partially separate or distinct current path through the abdomen. Preferably, the current source is adapted to provide the alternating current signal to each of the driving electrodes intermittently or at intervals and offset from one another; i.e., sequentially. The meter or sensor will preferably determine or measure the conductivity of the abdomen along each of the current paths between the electrodes periodically.

As noted above, the electrodes are adapted for application to opposite sides of an abdomen of the patient in a region of the stomach, pyloric antrum, duodenum and/or at a level of the kidneys.

In an embodiment, the method and system of the present disclosure measure a number of separate current paths in a “round-robin” fashion. A complete measurement of the multiple paths occurs several times per second. This involves a plurality of low frequency measurements of conductivity, a plurality of high frequency measurements of conductivity, and preferably also reference measurements of a local fixed (non-varying) internal resistance to confirm correct functioning of the system.

According to a further aspect, the disclosure provides electrodes designed for a system for assessing or evaluating gastrointestinal health, especially function, alteration in function, or dysfunction of a stomach, small intestine, or peritoneum, and particularly for assessing gastrointestinal motility of a patient. The electrodes comprise: at least one pair of electrodes adapted for application to an abdomen of the patient, and preferably to opposite sides of the abdomen, wherein at least one of the electrodes for supply of an electric current to the abdomen (e.g., as a driving electrode), and at least one of the electrodes is configured for receiving electric current along a current path through the abdomen (e.g., as a sensing electrode). The electrodes are designed for connection to a current source to provide a current signal to the electrodes, and for connection to a meter or a sensor to determine or measure a conductivity of the abdomen along the current path between the electrodes. As noted above, the output conductivity that is determined or measured between the electrodes is to be analysed or evaluated over a period of time to assess gastrointestinal health, and especially potential dysfunction in a region of the stomach, small intestine, or peritoneum. To this end, changes in measured conductivity over time can provide an indication of alteration in function or dysfunction of a stomach, small intestine, or peritoneum of the patient, and particularly gastrointestinal motility und function within this region.

The electrodes of the electrode pair will be specifically adapted to the system and method of the present disclosure. To this end, the calibration of the meter or sensor for determining or measuring the conductivity of the abdomen along the current path between the electrodes, and calibration of the processor for analysing and/or evaluating the conductivity of the abdomen determined or measured by the meter or sensor over a period of time to provide an assessment of gastrointestinal motility, particularly to detect function or dysfunction in a region of the stomach, small intestine, or peritoneum, will typically be very sensitive to variations in the electrodes. For this reason, the electrodes of the system and method of the disclosure will be standardised to minimise sources of variability that could confound measurements or test results obtained. For example, it is anticipated that the type of the electrode, its structure, size, and geometry, as well as its interaction with the skin of the patient (e.g., via a conductive gel pad), will be critical to reliable functionality of the system and method. For this reason, the electrodes are desirably standardised. It will be appreciated that features of electrode design for use on the human body, and especially for conductive application to human skin, are quite well-established in view of the various medical applications for such electrodes. An example in this regard are they electrodes for an electrocardiogram (ECG). In that particular case the electrodes are for receiving or detecting electrical activity of the heart, rather than for applying an electric current to the body, but the features of conductive connection to the skin are nevertheless relevant.

In an embodiment, the electrodes may be serialised or coded for compatibility with the processor and/or for compatibility with the meter or sensor of the system. In this way, the system of the disclosure may be adapted to operate only with electrodes that are “recognised” by and compatible with the system; i.e., for which the system has been designed and calibrated, thereby minimising sources of variability or errors in the patient measurements or test results. All electrodes may be structurally substantially identical.

According to a further aspect, the disclosure provides a method for assessing or evaluating intra-abdominal health of a patient, especially function, alteration in function, or dysfunction of a stomach, small intestine or peritoneum, particularly gastrointestinal motility, comprising:
storing electronic program instructions for controlling a controller; and
controlling the controller via the electronic program instructions, to perform the following operations:
   provide or apply a current signal to or between at least one pair of electrodes applied to an abdomen of the patient, wherein at least one of the electrodes supplies electric current to the abdomen and at least one of the electrodes receives electric current along a current path through the abdomen;
   determine or measure a conductivity of the abdomen along the current path between the electrodes, e.g., via a meter or sensor; and
   analyse the conductivity of the abdomen determined or measured over a period of time to provide an assessment of intra-abdominal health.

According to yet another aspect, there is provided a computer-readable storage medium on which is stored instructions that, when executed by a computing means, cause the computing means to perform a method according to the aspect of the present disclosure as hereinbefore described.

According to still a further aspect, the disclosure provides a computing means programmed to carry out the method according to the aspect of the present disclosure as hereinbefore described.

### BRIEF DESCRIPTION OF THE DRAWINGS

For a more complete understanding of the disclosure and advantages thereof, exemplary embodiments are explained in more detail in the following description with reference to the accompanying drawing figures, in which like reference signs designate like parts and in which:

FIG. 1 is an image of the abdomen or gut of a patient in transverse section showing anatomical structures of interest;

FIG. 2 is a schematic electrical model of the body illustrating the two main parts or components (i.e., extra-cellular and intra-cellular) in a given region of the body as a simple parallel circuit;

FIG. 3 is a schematic illustration of a system for assessing gastrointestinal motility of a patient in use according to an embodiment of the disclosure;

FIG. 4 is a schematic illustration of parts of a system for assessing gastrointestinal motility of a patient according to an embodiment of the disclosure; and

FIG. 5 is a flow diagram that schematically represents a method according to any of the embodiments of the present disclosure.

The accompanying drawings are included to provide a further understanding of the present disclosure and are incorporated in and constitute a part of this specification. The drawings illustrate particular embodiments and together with the description serve to explain the principles of the present disclosure. Other embodiments and many of the attendant advantages will be readily appreciated as they become better understood with reference to the following detailed description.

It will be appreciated that common and/or well understood elements that may be useful or necessary in a commercially feasible embodiment are not necessarily depicted in order to facilitate a more abstracted view of the embodiments. The elements of the drawings are not necessarily illustrated to scale relative to each other. It will also be understood that certain actions and/or steps in an embodiment of a method may be described or depicted in a particular order of occurrences while those skilled in the art will understand that such specificity with respect to sequence is not actually required.

### DETAILED DESCRIPTION OF THE EMBODIMENTS

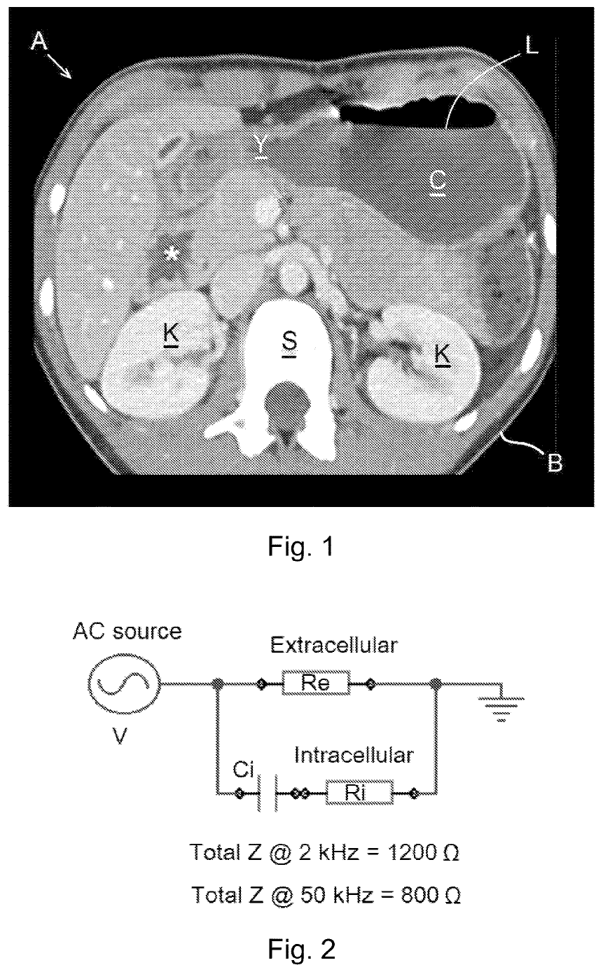

With reference firstly to FIG. 1 of the drawings, an abdomen or gut A of a patient body B is shown in transverse section. There are some locations within the abdomen A where the associated anatomical structures of the gastrointestinal tract are relatively invariant. These include the pyloric region Y of the stomach C, and the duodenum. Most other parts of the gastro-intestinal tract are able to move around within the abdomen A to some degree. The image of the gut A in transverse section in FIG. 1 shows the anatomical structures of interest, as well as the pancreas F. The section is taken at the level of the kidneys K (i.e., in a region of thoracic vertebra T12 to lumbar vertebra L2) and is shown looking up from the feet towards the head, with the patient's right side on the left-hand side of the image, and the spine S locate towards the bottom of the image. The stomach C can be seen here with some fluid level L in it, from which we can assume that the patient is in a supine position, i.e., lying on his/her back.

The flow of electrical current through the body is governed by the conductivity of the structures and substances located along the current path. The highest conductivity substances in the human body are typically extra-cellular fluids, such as urine, lymph, and blood, because these are predominantly comprised of salts in a solution of mostly water. The next most conductive substances are typically muscle and vascular organs like the liver or kidney. Bone, fat, and skin are the poorest electrical conductors. From the point of view of conductivity, there are two main parts within human tissue—namely, the intracellular matrix and extracellular matrix. The extracellular matrix/fluid includes the contents of the stomach or gut, blood, lymphatic fluid within tissue, and the like. The cellular membrane is a wall-like structure that encloses a cell and holds it together, with intracellular fluid or liquid being inside the cell. The cell membrane is comprised of a fat (lipid) with holes (pores) in it. While the fat layer of the cellular membrane has very poor conductivity, the liquid inside the cell is a saline solution and is generally of much higher conductivity. These two parts of the tissue (within the same region of the body) can thus be modelled as a simple parallel circuit, as illustrated in FIG. 2 of the drawings.

Referring to drawing FIG. 2, the path of an electric alternating current signal from an AC source V directed through the extracellular matrix can be modelled as a simple resistive circuit with resistor Re. The path of an electric alternating current signal through the intracellular matrix, on the other hand, is modelled by a capacitor Ci in series with a resistance Ri, where the capacitor Ci represents the cellular membrane. This means that a low frequency alternating current will involve a lower conductivity, because the low frequency alternating current (e.g., <2 kHz) can only pass through the extracellular matrix, whereas a high frequency alternating current (e.g., >50 kHz) will involve a higher conductivity over the same region, because some of the current will pass through the inside of the cells. The total impedance (Z) is shown in FIG. 2 for two alternating current frequencies. With this background, the system and method of the disclosure has been developed to employ a dual-frequency arrangement.

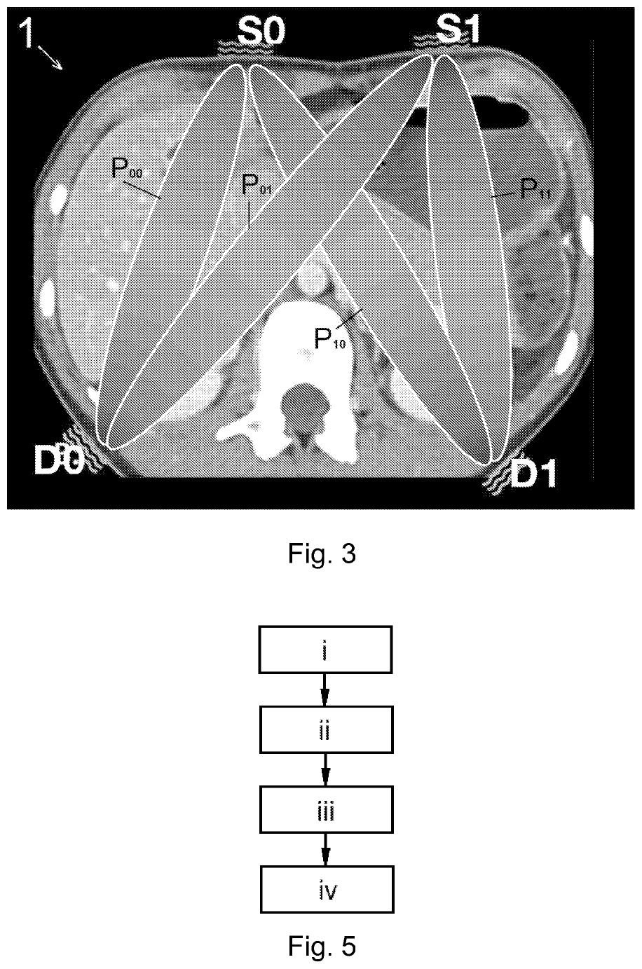

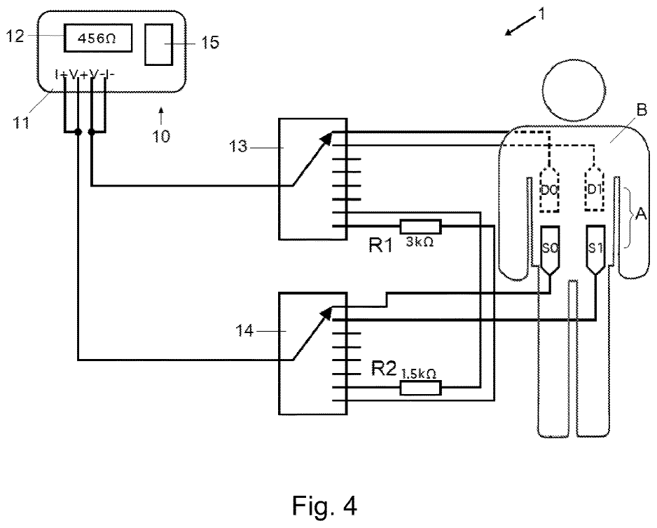

With reference to drawing FIGS. 3 and 4, a system 1 and an associated method pursuant to an embodiment of the disclosure will be described. The system 1 and the method are for measuring conductivity between electrodes D0, D1, S0, S1 placed and conductively applied on the skin at the back (posterior) and the front (anterior) sides of the abdomen A of a patient's body B as a means to assess health and activity of the gastrointestinal tract, and more particularly, to assess indirectly peristaltic movement of the gastrointestinal tract and movement of fluid across the pyloric sphincter, or between the pyloric antrum and the duodenum, and thereby to evaluate function or alteration in function of the stomach, the small intestine, or the peritoneum. As can be seen in FIG. 3, four electrodes D0, D1, S0, S1 are applied or placed on the abdomen A of the patient, including two drive electrodes D0, D1 for supplying an alternating electric current, which are applied to the back or posterior side of the patient, and two sensing electrodes S0, S1 for receiving alternating electric current along a current path through the abdomen, which applied to the front or anterior side of the patient. All of the electrodes D0, D1, S0, S1 are applied at about the level of the kidneys (i.e., in a region of thoracic vertebra T12 to lumbar vertebra L2).

The presumptive current paths (labelled “P”) are shown schematically in FIG. 3 as elliptical regions. As in FIG. 1, this view of the abdomen A is seen from the patient's feet, looking towards the head. The electrodes D0, D1, S0, S1 are connected in pairs, with each of the rear side (driving) electrodes D0, D1 arranged to form a pair with each of the front side (sensing) electrodes S0, S1. In this way, there are four electrode pairs D0S0, D0S1, D1S0, D1S1. As seen in FIG. 4, the system 1 comprises a device 10 for use in detecting function or dysfunction of the stomach, small intestine, or peritoneum, and especially for detecting gastrointestinal motility in this region of the patient's body B. The device 10 includes a current source 11 which is adapted for providing or applying an alternating current signal to or between each pair of the electrodes D0, D1, S0, S1 applied to the front and rear sides of the abdomen A of the patient's body, and a meter or sensor 12 for determining or measuring a conductivity of the abdomen A along the current path P₀₀, P₀₁, P₁₀, P₁₁ between each respective electrode pair. When current is applied between any pair of the respective electrodes D0, D1, S0, S1, the electrodes are also interrogated by the meter or sensor 12. The provision of the current signal from the current source 11 and/or the interrogation or sampling by the meter or sensor 12 occur(s) periodically at rates in a range of between 5 and 30 samples per second. The sampling at these rates enables the patient's cardiac signal to be eliminated from the measurements with higher sampling rates addressing this issue more effectively. Each electrode pair D0S0, D0S1, D1S0, D1S1 has both a low frequency alternating current signal of about 200 Hz and a high frequency alternating current signal of about 50 KHz applied intermittently and sequentially via the current source 11. The conductivity of each current path P₀₀, P₀₁, P₁₀, P₁₁ is measured for both the high frequency current signal and the low frequency current signal as a high frequency conductivity Oni and a low frequency conductivity σₗₒ. The offset conductivity σ_{offset} is determined as an offset at a time Tset that is set for starting assessment of the case, and the normal or overall conductivity is compared to this reference during the rest of the case assessment.

In general terms, the low frequency conductivity σₗₒ is subtracted from the high frequency conductivity Oni for each current path P₀₀, P₀₁, P₁₀, P₁₁ of the electrode pairs shown D0S0, D0S1, D1S0, D1S1. A normal conductivity or overall conductivity is then created for each path, which should be close to numerical zero, and the changes in the conductivity between the four paths P₀₀, P₀₁, P₁₀, P₁₁ is then observed over time.

σ_{offset(Tset)}=σ_{hi(Tset)}−σ_{lo(Tset)} (Equation 1)

σₒᵥₑᵣₐₗₗ=σₕᵢ−σₗₒ−σoffset(Tset) (Equation 2)

It is expected that the conductivity will change slowly over a period of some tens of seconds, in a sequential manner (e.g., between 10 seconds and 30 seconds). As the conductivity measured or determined for the path P₀₁ between the electrode pair D0S1 increases, it may be expected that the stomach has forced more fluid into the pyloric antrum. And as this conductivity measured or determined for the path P₀₁ between the electrode pair D0S1 increases, showing fluid in the duodenum, it may be expected that the conductivity measured or determined for the path P₀₀ between electrode pair D0S0 should decrease. This is because the fluid has now been forced from the pyloric antrum across the sphincter, into the duodenum.

The electrode pair D1S1 measures the conductivity across the stomach, which in this particular case is likely to be less than the conductivity measured by the electrode pair D0S1, as there is gas in the top part of the stomach (i.e., assuming the patient is in a supine position). However, this lower conductivity measurement at D1S1 could also simply be an indication that the electrode S1 is becoming dislodged. The conductivity measured or determined by each of the two electrode pairs D0S1 and D1S1 would both drop if this were to occur. Where changes in the conductivity measured or determined by the respective electrode pairs D0S0, D0S1, D1S0, D1S1 are due to fluid movements in the body B, however, one would expect a complementary (paradoxical) conductivity change with a value of conductivity between one electrode pair rising and between the other pair falling. The same should occur with the other electrode pairs D0S0 and D1S0. If the conductivities measured or determined between both of these pairs of electrodes D0S0, D1S0 should fall, electrode failure or disconnect may be suspected. But if the conductivity between one pair rises while the conductivity between the other electrode pair falls, a movement of fluid across the pyloric sphincter may be concluded, which then provides an indication or a potential indication of gastrointestinal forward transit. Evidence of transit can then be flagged for confirmation by a medical professional, who may then examine the record, assess the evidence, and confirm or refute according to their clinical judgement.

The conductivity between each of the electrode pairs D0S0, D0S1, D1S0, D1S1 for the four separate current paths P₀₀, P₀₁, P₁₀, P₁₁ is measured in repeating sequence in a “round-robin” fashion, with one complete measurement of each of the four current paths at least every 200 msec. This then, in turn, corresponds to at least five complete measurements per second. This sequence involves four low frequency measurements of the conductivity across each electrode pair, four high frequency measurements of the conductivity across each electrode pair, and two measurements of a local internal fixed or invariant resistor to ensure correct device function in each 200 msec cycle. As noted above, higher sampling rates enables the patient's cardiac signal to be eliminated from the measurements more effectively, and a regime of up to 30 complete measurements per second has been trialed (i.e., with one complete measurement of the four current paths P₀₀, P₀₁, P₁₀, P₁₁ every 33 msec).

With reference to FIG. 4 of the drawings, a simple circuit diagram is shown to illustrate the system 1 of a preferred embodiment. The system 1 has driving electrodes D0, D1 (in broken lines representing their positioning hidden at the rear or posterior side of the patient body B) connected in circuit with the sensing electrodes S0, S1, with all of the electrodes D0, D1, S0, S1 being connected to the current source 11 via two eight-channel multiplexers 13, 14 for switching between the respective electrode pairs D0S0, D0S1, D1S0, D1S1. The two resistances R1, R2 are provided as local internal fixed or invariant resistors for calibration and on-going reference or cross-checking during use of the system 1. The device 10 comprises the current source 11 for providing an AC signal to the electrode pairs D0S0, D0S1, D1S0, D1S1, as well as the meter or sensor 12 for interrogating those electrode pairs periodically at rates in a range of between 5 and 30 samples per second to measure or determine conductivity along each of the current paths P₀₀, P₀₁, P₁₀, P₁₁ of the respective electrode pairs D0S0, D0S1, D1S0, D1S1. A processor 15 (with memory) is incorporated in the device 10 for recording or storing the values of conductivity that are measured or determined by the meter 12 over a patient monitoring period. Furthermore, the processor 15 is adapted to analyse the conductivity of the abdomen A determined or measured by the meter or sensor 12 over that period of time, and particularly changes in the conductivity determined or measured along the respective current paths P₀₀, P₀₁, P₁₀, P₁₁, to assess and/or detect function or alteration in function of the stomach, small intestine, or peritoneum.

Finally, referring to FIG. 5 of the drawings, a flow diagram is shown to illustrate schematically the steps in a method of assessing or evaluating function, alterations in function, or dysfunction of the stomach, small intestine, and peritoneum, and especially gastrointestinal motility, of a patient according to the embodiments of the disclosure described above with respect to FIGS. 1 to 4. In this regard, the first box i of FIG. 5 represents the step of applying electrodes D0, D1, S0, S1 to the patient's abdomen, the electrodes including at least one electrode D0, D1 at applied a rear of the abdomen and at least one electrode S0, S1 applied at a front of the abdomen for electric current that passes or is conducted along a current path P through the abdomen. In the particular example, the two electrodes D0, D1 are applied spaced apart on the posterior side of the abdomen and the two electrodes S0, S1 are applied spaced apart on the anterior side of the abdomen, with all of the electrodes D0, D1, S0, S1 applied at the level of the kidneys. The second box ii represents the step of providing an alternating current signal to the at least one electrode D0, D1, with the current signal provided intermittently or at intervals to each of the electrodes D0, D1 and sequentially or offset from one another. The third box iii then represents the step of determining or measuring conductivity of the abdomen along each current path P₀₀, P₀₁, P₁₀, P₁₁ between the electrode pairs D0S0, D0S1, D1S0, D1S1 formed by the at least one rear electrode D0, D1 and the at least one front electrode S0, S1. The final box iv in FIG. 5 of the drawings represents the step of evaluating changes in the conductivity of the abdomen determined or measured between the electrode pairs D0S1, D1S0, D1S1 over a period of time, whereby the changes in the conductivity of the abdomen over a period of time provide an indication of potential alterations in function or dysfunction of the stomach, small intestine, and peritoneum, and especially of gastrointestinal motility of the patient.

Although specific embodiments of the disclosure are illustrated and described herein, it will be appreciated by persons of ordinary skill in the art that a variety of alternative and/or equivalent implementations exist. It should be appreciated that each exemplary embodiment is an example only and is not intended to limit the scope, applicability, or configuration in any way. Rather, the foregoing summary and detailed description will provide those persons skilled in the art with a convenient road map for implementing at least one exemplary embodiment, it being understood that various changes may be made in the function and arrangement of elements described in an exemplary embodiment without departing from the scope as set forth in the appended claims and their legal equivalents. Generally, this application is intended to cover any adaptations or variations of the specific embodiments discussed herein.

The methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g. a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

It will also be appreciated that, unless the context requires otherwise, the terms “comprise”, “comprising”, “include”, “including”, “contain”, “containing”, “have”, “having”, and any variations thereof, used in this document are intended to be understood in an inclusive (i.e. non-exclusive) sense, such that the process, method, device, apparatus, or system described herein is not limited to the features, integers, parts, elements, or steps recited but may include other features, integers, parts, elements, or steps not expressly listed and/or inherent to such process, method, device, apparatus, or system. Furthermore, the terms “a” and “an” used herein are intended to be understood as meaning one or more unless explicitly stated otherwise. In addition, reference to positional terms, such as “lower” and “upper”, used in the above description are to be taken in context of the embodiments depicted in the figures, and are not to be taken as limiting the disclosure to the literal interpretation of the term but rather as would be understood by a skilled addressee in the appropriate context.

1.-32. (canceled)

33. A system for evaluating or detecting function or alteration in function of a small intestine, stomach, or peritoneum of a patient, comprising:
at least one pair of electrodes adapted for application to the patient's skin at opposite sides of an abdomen of the patient for supplying and receiving an electric current along at least one current path through the abdomen;
a current source for providing or applying a current signal to or between the electrodes, wherein the current source provides a high frequency alternating current signal of more than about 10 kHz;
a meter or a sensor for determining or measuring a conductivity of the abdomen along the current path between the electrodes; and
a processor for analysing the conductivity of the abdomen determined or measured by the meter or sensor over a period of time to detect function and alterations in function of the stomach, small intestine, or peritoneum.

34. The system according to claim 33, wherein the current source is adapted to provide an alternating current signal to the electrodes at a current at least one pair of electrodes at a current of less than or equal to about 100 μA.

35. The system according to claim 33, wherein the sensor is adapted to determine or measure the conductivity of the abdomen between the at least one pair of electrodes at least 5 times per second.

36. The system according to claim 33, wherein said at least one pair of electrodes comprises four electrodes, including two electrodes adapted to be applied to the patient's skin spaced apart from one another by a distance in a range of about 100 mm to about 500 mm on a first side of the abdomen and two electrodes adapted to be applied to the patient's skin spaced apart from one another by a distance in a range of about 100 mm to about 500 mm on a second opposite side of the abdomen.

37. The system according to claim 36, wherein each of the second side electrodes forms an electrode pair with each of the first side electrodes and receives electric current from each of the first side electrodes along a separate or distinct current path through the abdomen.

38. The system according to claim 36, wherein the current source is adapted to provide the alternating current signal to each electrode intermittently or at intervals and sequentially or offset from one another.

39. The system according to claim 36, wherein the meter or sensor is adapted to determine or measure periodically the conductivity of the abdomen along each of the current paths between the electrode pairs sequentially.

40. The system according to claim 33, wherein the electrodes are adapted for application to opposite sides of the abdomen in a region of the patient's stomach, pyloric antrum, duodenum, and/or kidneys.

41. The system according to claim 33, further comprising a three-axis accelerometer to be placed on the patient to detect gross body movements in order to indicate a resting body and to account for same in an analysis of the conductivity of the abdomen measured or determined over the period of time.

42. A method of evaluating or detecting function or alteration in function of a small intestine, stomach, or peritoneum, and especially gastrointestinal motility within this region, of a patient, comprising steps of:
applying electrodes to the patient's skin at opposite sides of an abdomen of the patient in a region of the patient's stomach, pyloric antrum, duodenum and/or at a level of the patient's kidneys, the electrodes including at least one electrode for supplying an electric current to the abdomen of the patient and at least one electrode for receiving an electric current along a current path through the abdomen of the patient;
providing an alternating current signal of more than about 10 kHz to the electrodes; and
determining or measuring conductivity of the abdomen along the current path between the electrodes, wherein changes in the measured or determined conductivity of the abdomen over a period of time provide an indication of function or alteration in function of the small intestine, stomach, or peritoneum.

43. The method according to claim 42, wherein the step of applying electrodes to the patient's skin at opposite sides of an abdomen of the patient comprises applying at least two electrodes spaced apart from one another on a first side of the abdomen and applying at least two electrodes spaced apart from one another on a second side of the abdomen.

44. The method according to claim 43, wherein each second side electrode forms an electrode pair with each first side electrode and receives electric current from each of the first side electrodes along a separate or distinct current path through the abdomen.

45. The method according to claim wherein the step of providing the current signal includes providing the current signal intermittently or at intervals to each of the electrodes and sequentially or offset from one another.

46. The method according to claim 42, wherein the step of determining or measuring the conductivity of the abdomen includes determining or measuring the conductivity between the electrodes on a periodic basis over the period of time.

47. The method according to claim 42, wherein the step of determining or measuring the conductivity of the abdomen is performed at least 5 times per second.

48. The method according to claim 42, wherein the step of providing a current signal includes providing the current signal to the electrodes at a current of less than or equal to about 100 μA.

49. A method of evaluating or detecting function or alteration in function of a small intestine, stomach, or peritoneum, and especially of assessing gastrointestinal motility within this region, of a patient, comprising steps of:
applying electrodes to the patient's skin at opposite sides of an abdomen of the patient for supplying and receiving electric current along at least one current path through the abdomen, especially in a region of the stomach, pyloric antrum, duodenum and/or kidneys of the patient;
providing an alternating current signal of more than about 10 kHz to the electrodes; and
determining or measuring conductivity of the abdomen along the current path between the electrodes;
analysing changes in the conductivity of the abdomen measured or determined along the current path over a period of time to provide an indication of function or dysfunction of the small intestine, stomach, or peritoneum.

50. The method according to claim 49, wherein the step of applying electrodes to the patient's skin at opposite sides of the abdomen of the patient comprises applying at least two electrodes spaced apart from one another on a first side of the abdomen and applying at least two electrodes spaced apart from one another on a second side of the abdomen.

51. A method for evaluating or detecting function or an alteration in function of a stomach, small intestine or peritoneum, especially gastrointestinal motility in this region, of a patient, comprising:
storing electronic program instructions for controlling a controller; and
controlling the controller via the electronic program instructions, to perform the following operations:
provide or apply a current signal of more than about 10 kHz to or between at least one pair of electrodes applied to the patient's skin on an abdomen of the patient, wherein at least one of the electrodes supplies electric current to the abdomen of the patient and at least one of the electrodes receives electric current along a current path through the abdomen of the patient;
determine or measure a conductivity of the abdomen along the current path between the electrodes; and
analyse the conductivity of the patient's abdomen determined or measured over a period of time to evaluate or detect the function or the alteration in function of a stomach, small intestine, or peritoneum.

52. The method according to claim 51, wherein the operation to determine or measure a conductivity of the abdomen along the current path between the electrodes is performed at least 5 times per second.
